(19) United States
(12) Patent Application Publication
(10) Pub. No.: US 2004/0241825 A1
(43) Pub. Date: Dec. 2, 2004

(54) Methods for generating libraries of therapeutic bacteriophages having desired safety characteristics and methods for labeling and monitoring bacteriophages

(76) Inventors: Rosemonde Mandeville, Ste Therese (CA); Beatrice Allain, Montreal (CA); Kishore Murthy, Dollards des Ormeaux (CA)

Correspondence Address: DARBY & DARBY P.C., P. O. BOX 5257, NEW YORK, NY, 10150-5257, US

(21) Appl. No.: 10/475,195

(22) PCT Filed: Apr. 19, 2002
(86) PCT No.: PCT/CA02/00569

Related U.S. Application Data
(60) Provisional application No. 60/284,517, filed on Apr. 19, 2001.

(51) Int. Cl. C12N 1/20

(52) U.S. Cl. 435/252.3

(57) ABSTRACT

The present invention is concerned with genetically labeled bacteriophages, and with methods for preparing and using the same. Genetically labeled phages may easily be distinguished from non-labeled phages. The present invention also relates to methods for selecting therapeutic bacteriophages lysing pathogenic bacteria without cross-reacting with non-pathogenic bacteria. This method permits the selection of phages which are highly specific to given pathogenic bacteria. The present invention is also concerned with methods for evaluating bacteriophage susceptibility to external genetic modifications in order to provide bacteriophages that are safe and highly specific for use in the prevention, treatment and/or control of bacterial infections or contamination in plants, animals, and humans, as well as environmental cleanup and sanitation.

### RELATED APPLICATION

This application claims priority of U.S. Provisional Application 60/284,517 filed Apr. 19, 2001, the disclosure of which is incorporated by reference herein in its entirety.

### BACKGROUND OF THE INVENTION

A) Field of the Invention

The present invention is concerned with genetically labeled bacteriophages, and with methods for preparing and using the same. The present invention also relates to methods for selecting therapeutic bacteriophages lysing pathogenic bacteria without cross-reacting with non-pathogenic bacteria such as beneficial bacteria living in symbiosis with a living host. The present invention is also concerned with methods for evaluating bacteriophage susceptibility to external genetic modifications in order to provide bacteriophages that are safe and highly specific for use in the prophylactic and therapeutic treatment of bacterial infections in plants, birds, livestock, and humans.

B) Brief Description of the Prior Art

Drug resistant pathogens are becoming a growing problem both in humans and in the livestock industry. In humans, infectious diseases are the second leading cause of death accounting for 25% of total mortality worldwide. Bacterial infections are one of the leading contributors to this group. Bacterial resistance to antibiotics has now emerged as one of the major public health threats internationally. In North America alone, more than 60% of hospital acquired infections are caused by drug-resistant bacteria. The extra cost of treating these infections is inducing a lot of financial strain on the world economies and causing loss of human lives.

In the livestock sector, farmers are bearing huge economic losses due to antibiotic resistant bacterial infections, and in most farms, the incidence of antibiotic resistant organisms is continuously on the rise. It is estimated that over 60 to 80% of all the cattle, sheep, swine and poultry in the U.S. receive antibiotics regularly mainly to promote growth. This overuse of antibiotics in livestock is one of the contributing causes for the emergence of antibiotic resistance both in livestock and humans.

Phages as antibacterial are a very attractive alternative to address the present problem of antibiotic resistance. Bacteriophages are ubiquitous in nature and thrive wherever their host bacteria are present. They do not infect unrelated bacteria or mammalian cells. Phage therapy has several advantages over conventional antibiotic therapy. Some of these advantages include: a relatively high specificity, a single dose of phage treatment is often sufficient, and development of resistance to phages is approximately 10 fold slower than that for antibiotics.

There are however important problems of safety and efficacy associated with the therapeutic use of phages. First, when therapeutic phages are used for treatment purposes, it is very difficult, and most of the time impossible, to identify and monitor therapeutic phages used in the treatment and distinguish them from those naturally found in the host in which phages are administered, as well as, those coming from the outside environment. Second, it is not rare that phages isolated against pathogenic bacteria also cross-react with non-pathogenic bacteria living in symbiosis with the host. Third, bacteriophages are subject to external genetic modifications, (e.g. bacterial genes coding for toxins may be incorporated into the genome of phages) which raise some concerns, particularly if such bacteriophages are to be used in phage therapy protocols.

There is therefore a need for novel methods and approaches for selecting and labeling phages to circumvent the problems known in the art. The present invention fulfils these needs and also other needs which will be apparent to those skilled in the art upon reading the following specification.

### SUMMARY OF THE INVENTION

i) Genetically Labeled Bacteriophages

According to a first aspect, the present invention relates to phages genetically modified for incorporating into their genome a detectable signature label so that these labeled phages may be easily distinguished from other non-labeled phages. According to an embodiment of the invention, this object is achieved with a genetically labeled bacteriophage, the bacteriophage having a genetically modified genome comprising an exogenous nucleic acid label sequence, the label sequence consisting of an assemblage of nucleotides forming a detectable signature sequence having a non-functional coding function. Preferably, the exogenous nucleic acid sequence is inserted into a non-coding region of the genome of the phage.

According to a related aspect, there is provided a method for producing a genetically labeled bacteriophage. The method comprises the steps of:

providing a biologically active bacteriophage with a genome;

providing an exogenous detectable nucleic acid sequence to be used as a label; and

inserting the exogenous detectable nucleic acid sequence into a suitable region of the genome of the bacteriophage so that the insertion of the exogenous detectable nucleic acid sequence does not substantially negatively affect the bacteriophage biological activity.

According to another related aspect, there is provided a method for producing a genetically labeled bacteriophage which can be distinguished from non-labeled bacteriophages. The method comprises the steps of;

providing a biologically active bacteriophage with a genome;

providing an exogenous detectable nucleic acid sequence to be used as a label, the label sequence consisting of an assemblage of nucleotides forming a detectable signature sequence having a non-functional coding function; and

inserting the exogenous detectable nucleic acid sequence into a suitable region of the genome of the bacteriophage so that said insertion does not substantially negatively affect said bacteriophage biological activity; whereby detection of the exogenous detectable nucleic acid sequence permits to distinguish genetically labeled bacteriophages from non-labeled bacteriophages.

The present invention also encompasses the labeled bacteriophage which have been produced according to one of the above mentioned methods.

According to another related aspect, there is provided a method of phage therapy, wherein labeled bacteriophages as defined hereinbefore are administered to a host.

According to a further related aspect, there is provided a method for determining or monitoring the presence of bacteriophages in a sample, the method comprising the step of detecting the presence in the sample of labeled bacteriophages as defined previously.

Yet, in a further related aspect, the invention provides a method for the prevention, treatment and/or control of a bacterial infection or contamination in a host, comprising the step of administering to said host a plurality of biologically active and genetically modified bacteriophages as defined hereinbefore.

According to a further related aspect, there is provided a method for confirming the absence of bacteriophages in an animal carcass derived from a living animal subjected to a treatment of phage therapy with labeled bacteriophages as defined hereinbefore, the method comprising the steps of:

obtaining a sample from the carcass; and

assaying the sample obtained for detecting therein the presence or absence of labeled bacteriophages.

Absence of detection for labeled bacteriophages is then indicative that the carcass is free from these labeled bacteriophages used for the treatment of the host.

ii) Non-Cross Reacting Bacteriophages

According to a second aspect, the present invention relates to methods for isolating and selecting phages that react against pathogenic bacteria without cross-reacting against non-pathogenic bacteria living in symbiosis with the host. Such phages are particularly useful for therapeutic purposes.

More particularly, the invention provides a method for selecting bacteriophages capable of lysing a selected strain of pathogenic bacteria without lysing bacteria from a selected non-pathogenic strain. This method comprises the steps of:

a) contacting under conditions suitable for lysis: i) bacteriophages capable of lysing a selected pathogenic strain of bacteria, and ii) bacteria from the selected pathogenic strain;

b) isolating, from the bacteriophages of step a), bacteriophages capable of lysing at least some of the pathogenic bacteria they have been contacted with;

c) contacting, under conditions suitable for lysis, bacteriophages isolated at step b) with a selected strain of non-pathogenic bacteria for which lysis is undesirable, wherein the non-pathogenic bacteria and the pathogenic bacteria belong to a common genus; and

d) selecting, from the bacteriophages of step c), bacteriophages which do not lyse more than 5% of the non-pathogenic bacteria they have been contacted with.

ii) Bacteriophages Susceptibility to External Genetic Modification

According to a third aspect, the present invention aims to provide bacteriophages which have been selected for their inability or non-susceptibility to suffer from external genetic modification into their genome such as integration of bacterial genetic sequence coding for toxins.

Accordingly, the present invention provides a method for evaluating bacteriophage susceptibility to external genetic modifications. According to a first embodiment, the method comprises the steps of:

a) providing a first and a second pool of bacteriophages, the first and second pools both comprising a plurality of an identical type of bacteriophages for which susceptibility to genetic modifications is to be evaluated;

b) contacting bacteriophages from the first pool with bacteria under suitable conditions and for a sufficient period of time to allow the bacteria to cause genetic modifications to the bacteriophages if this type of bacteriophages have such a susceptibility;

c) isolating bacteriophages from the bacteriophages contacted at step b);

d) digesting separately, with a plurality of restriction enzymes, genetic material from the bacteriophages from the second pool and genetic material of bacteriophages isolated at step c), thereby obtaining a restriction digestion pattern for bacteriophages of the first and second pools; and

e) comparing the restriction digestion patterns of step d), wherein a difference between these restriction digestion patterns is indicative that the type of bacteriophages evaluated is susceptible to genetic modifications.

According to another embodiment, the method for evaluating bacteriophage susceptibility to external genetic modifications comprises the steps of:

a) providing bacteriophages for which susceptibility to genetic modifications is to be evaluated;

b) contacting these bacteriophages with bacteria under suitable conditions and for a sufficient period of time to allow the bacteria to cause genetic modifications to the bacteriophages if this type of bacteriophages have such a susceptibility;

c) isolating bacteriophages from the bacteriophages contacted at step b);

d) digesting with a plurality of restriction enzymes, genetic material from the bacteriophages isolated at step c), thereby obtaining a restriction digestion pattern for the isolated bacteriophages; and

e) comparing the restriction digestion pattern of step d) with a control restriction digestion pattern obtained from phages not contacted with the bacteria, wherein a difference between these restriction digestion patterns is indicative that the type of bacteriophage evaluated is susceptible to genetic modifications.

An advantage of the present invention is that it allows the selection of phages which are highly specific to given pathogenic bacteria. According to the invention, it is also possible to generate libraries of phages that are safe, guaranteed to be non-toxic and highly specific for use in the prophylactic and therapeutic treatment of bacterial infections in a recipient in need thereof. It is also possible, according to the present invention, to genetically label phages with a “secret” signature sequence without interfering with normal phage functions, and use this signature sequence to easily identify labeled-phages from non-labeled phages.

Other objects and advantages of the present invention will be apparent upon reading the following non-restrictive description made with reference to the accompanying drawings

### BRIEF DESCRIPTION OF THE DRAWINGS

FIG. 1 is a picture of a restriction digest pattern of a Salmonella phage. The phage was digested as described in Example 2 with EcoRI, EcoRV, HindIII, PstI and SalI. The restriction digestion pattern is presented along with undigested phage DNA. M1 and M2 represent DNA molecular weight markers.

FIG. 2 is a picture of a restriction digestion pattern of eight different Salmonella phages that were digested with EcoRI and HindIII as described in Example 2. Letters A to H represent different classes of phages digested. M1 and M2 represent DNA molecular weight markers.

FIG. 3 is a picture of a HaelI restriction digest pattern of a Salmonella phage contacted or not contacted with different strains of E. coli and Salmonella as described in Example 2. Lane 1: Phage control DNA; Lanes 2 and 3: DNA from samples at Day 5; Lanes 4 and 5: DNA from samples at Day 7. Lanes 3 and 5 represent samples incubated with the bacterial pool and Lanes 2 and 4 their respective phage controls incubated in the amplification media. Lane M represents DNA molecular weight markers.

### DETAILED DESCRIPTION OF THE INVENTION

A) General Overview of the Invention

The present invention is concerned with phages comprising into their genome an exogenous “signature” sequence (label), with methods of genetically labeling, and methods of using such phages.

The present invention is also concerned with methods for generating libraries of biologically safe therapeutic bacteriophages using selection criteria and approaches different from those known in the art.

According to the present invention, it is possible to construct libraries of bacteriophages for various causative pathogens based on the cross-reaction characteristics of the phages with both pathogenic and non-pathogenic bacteria, such as bacteria living in symbiosis with a living host. Phages in these libraries are non-toxic when administered to a host, and do not cross-react with non-pathogenic bacteria. The phage libraries of the invention are particularly useful for developing a number of phages against numerous pathogens and capable to recognize different receptors on the bacterial surface. Once such libraries are constructed, different individual phages or different combinations of these phages can be used to prevent, treat and control bacterial infections and contamination. Another advantage of the libraries of the present invention is that if one phage in a group is found to be less efficacious in a particular treatment, a second phage having different characteristics can be immediately used to replace it in the treatment.

The words “phage” and “bacteriophage” are used herein equally. Furthermore, the present invention is not restricted to specific phages. Although the methods described refer to phages with a genome comprising double stranded DNA, the approaches described herein can also be employed for single stranded DNA and RNA phage genomes using alternative standard molecular biology protocols (Current protocols in molecular biology, 1995). A person skilled in the art could apply without undue experimentation the principles formulated herein to any type of phages, both DNA and RNA. A non-restrictive list of phages that could be used according to the invention includes phages capable of infecting bacterial organisms selected from the group consisting of: Actinobacillii, Actinomyces, Aeromonas, Archaebacteria, Agrobacteria, Aromabacter, Bacilli, Bacteriodes, Bifidobacteria, Bordetella, Borrelii, Brucella, Burkholderia, Calymmatobacteria, Campylobacter, Capnocytophaga, Citrobacter, Chlamydia, Clostridium, Coccus, Coprococci, Corynebacterium, Cyanobacter, Enterobacter, Enterococci, Eubacteria, Escherichia, Francisella, Helicobacter, Hemophilii, Hidradenitis suppurativa, Lactobacilli, Lawsonia, Legionella, Leptospirex, Listeria, Klebsiella, Mycobacterium, Mobiluncus, Neisserii, Nomerans, Pasteurella, Prevotella, Pneumococci, Propionibacteria, Proteus, Pseudomonas, Pyrococci, Salmonella, Selemonas Serratia, Shigella, Streptococci, Staphylococci, Streoticiccys, Succinimonas, Treponema, Veillonella, Vibrio, Woinella, Xanthomonas, and Yersinia

B) Phages Genetically Modified for Incorporating a Genetic Label and Method for Producing the Same

According to a first aspect of the invention, it is provided genetically labeled bacteriophage, preferably therapeutic phages, the bacteriophage having been genetically modified so that their genome incorporates an exogenous genetic “signature” label. The exogenous nucleic acid label sequence consists of an assemblage of nucleotides forming a detectable signature sequence having a non-functional coding function. A major advantage with such labeled phages, is that they may be easily identified, from other non-labeled phages, solely by a user knowing the identity of the label.

The label is a DNA or a RNA sequence depending on the phage type to be labeled (DNA or RNA type of phage). The label may have from about 5 to about 500 nucleotides, preferably from about 10 to about 50 nucleotides and more preferably from about 10 to about 25 nucleotides. Preferably also, the phage genome incorporates a single label. More preferably, the incorporation is stable. However, it is also possible to insert in the phage genome a plurality (from 2 to 10 and even more) of identical or different labels. Incorporation of a single and relatively short sequence is advantageous since such genome modification is less likely to interfere with the normal functions of the phage. Indeed, it is known in the art that the introduction of large sequences in the genome of a given host (phage, bacteria, yeast) may interfere with the stability of the host genome and host functions.

As mentioned previously, it is highly preferable, according to the present invention, that the label be a non-functional nucleic acid sequence, and that it be introduced into a non-coding region of the genome of a phage in order to minimize the risks of interfering with normal functions of the phage. However, it would be clear for a person skilled in the art that the exact sequence of the label(s) is not important and that the site(s) where the label(s) is to be inserted is not critical either. Indeed, it is conceivable that, under certain circumstances, a functional coding sequence inserted into a coding region of the genome of a phage would have minimal and even no effects on a phage normal functions. Therefore, instead of sequencing the genome of the phage for selecting the site where the label is to be inserted, a person skilled in the art could simply use random integration techniques and then screen the genetically modified phages in order to confirm that the phages have incorporated the label into their genome and that these labeled phages are functional.

The label is advantageously a synthetic oligonucleotide or a PCR generated fragment, depending on the label as well as its site of insertion. Preferably, the label sequence is cloned into a suitable region of the phage genome. Suitable cloning sites and suitable region can be established by carefully analyzing the sequence of the phage genome. If necessary, suitable cloning sites may be engineered into the label via its nucleic acid sequence. Cloning of the label into the phage genome can be done according to standard molecular biology protocols (Current protocols in molecular biology, 1995, chapter 3).

Advantages of the labeled phages of the invention are numerous: 1) different sequences (labels) may be used for different types of phages since the number of suitable sequences is almost infinite; 2) genetic modification in the non-coding region of a phage genome will not alter the properties of the phage: accordingly a labeled phage would be undistinguishable in its biological activity from a corresponding non-labeled phage; 3) by knowing the identity of the “secret” label, a user is able to easily confirm the identity of the phage and differentiate it from other similar phages, whereas a user not aware of the identity of the label will not be able to differentiate the labeled phages; and 4) the exogenous detectable nucleic acid sequence or label is detectable using many other well known methods and techniques such as PCR assays (e.g., conventional PCR (preferred), RT-PCR, Real-time PCR), sequencing the region into which the label has been inserted, biosensors, hybridization techniques (e.g. oligonucleotide hybridization, RNAse protection assay, enzyme-based in-situ hybridization methods), gel-shift assays, micro-analytical devices (e.g. microchips), high-throughput simultaneous test arrays (microarrays, gene chips), dipstick devices, dyes and labels.

Advantageously, the labeled phages may be used to confirm the absence of phages in the meat going to the market, pursuant phage therapy in animals raised for human consumption purposes (an issue similar to the issue of presence of residues from antibiotics in the meat at commercialization). For instance, monitoring of labeled phages by PCR will be much more resource-efficient and can be used on a routine basis to confirm the absence of phages in animal carcasses (with or without skin). Obtaining a negative result could be used as a criterion for marketability of the meat from animals receiving such treatment. The presence of a signature label on the phages will help in simplifying the process of monitoring phage residues and will also address the issue of safety of this anti-infective approach.

Therefore, the present invention is concerned with a method for the preventive or therapeutic control of a bacterial infection or contamination in a host, the method comprising the step of administering to a host a plurality of biologically active and genetically modified bacteriophages as defined hereinbefore. Preferably, the method further comprises the step of monitoring the presence of the bacteriophages in the host. More preferably, the monitoring step consists of detecting the exogenous nucleic acid label sequence of the phages. A variety of host could be treated according to this method, including but not limited to mammals (such as humans, livestock, domestic animals, wild animals), fish, sea food (mollusks, crustaceans, etc), birds, insects, invertebrates, reptiles, algae, plants (such as vegetables, fruits, trees). The method of the invention could also be adapted for environmental cleaning and sanitation of open water bodies, wells, and the like.

According to a preferred embodiment, the host consist of a human subject and the monitoring step is performed on feces, skin, body cavities, blood, urine samples or other body fluids from the treated host.

According to another preferred embodiment, host consist of a slaughtered animal which has been raised for human consumption purposes (including but not limited to pig, cattle, horses, chicken, turkey, rabbits, and wild animals such as bisons, ostrichs, deers, caribous, mooses, etc.), and the monitoring step is performed on sample taken from the carcass of said slaughtered animal. More preferably, the biologically active and genetically modified bacteriophages are administered to the animal about 20 days to about 6 hours before slaughtering. For monitoring purposes, the sample to be collected may be a sample from open cavities, body fluids (e.g., blood, urine, saliva, bronchial lavages, feces), solid organs (e.g. kidney, liver, lungs, brain, tongue), muscles and skin.

In a related aspect, the present invention is concerned with a method for confirming the absence of bacteriophages in an animal carcass derived from a living animal subjected to a treatment with phage therapy using labeled bacteriophages as defined hereinbefore. The method comprises the steps of obtaining a sample from the carcass; and assaying this sample for detecting the presence or absence of the labeled bacteriophages; whereby, the absence of detection of these labeled bacteriophages is indicative that the carcass is free from labeled bacteriophages. Typically, the detection simply consists of detecting the exogenous nucleic acid label sequence of the labeled bacteriophages is a sample as defined hereinabove.

Several other uses may be envisaged for the labeled phages of the invention. For instance, during phage therapy, labeled phages could be administered to distinguish the phages coming from the therapy from those coming from the patient or from the environment.

Regulatory authorities, such as U.S. FDA, having approved specific phages for therapeutic purposes, could use the “secret” label in these phages to control the quality of the phage preparations and keep track of the use and release of the phages in the environment. Similarly, any company commercializing phage preparations could also label its phages according to the present invention to check whether there is any unauthorized use of the phages being sold.

Labeled phages could also be used in environmental cleanup, sanitation and various environmental studies, particularly for monitoring purposes. Upon using the labeled phages of the invention for prophylactic or therapeutic purposes, it will be easy to determine the contribution of the labeled phages to the total pool of similar phages found in the environment. This data will help prove the insignificant contribution of the therapeutically used phages to the total pool of phages found in the environment.

C) Selection of Phases Reacting with Pathogenic Bacteria but Not Cross-Reacting with Non-Pathogenic Bacteria

According to a second major aspect of the invention, there is provided a method for selecting bacteriophages capable of lysing a selected strain of pathogenic bacteria without lysing bacteria from a selected non-pathogenic strain. This method comprises the steps of:

a) contacting under conditions suitable for lysis: i) bacteriophages capable of lysing a selected pathogenic strain of bacteria, and ii) bacteria from the selected pathogenic strain;

b) isolating, from the bacteriophages of step a), bacteriophages capable of lysing at least some of the pathogenic bacteria they have been contacted with;

c) contacting, under conditions suitable for lysis, bacteriophages isolated at step b) with a selected strain of non-pathogenic bacteria for which lysis is undesirable; and

d) selecting, from the bacteriophages of step c), bacteriophages which do not lyse more than 5% of the non-pathogenic bacteria they have been contacted with.

Preferably, the original phage preparation contacted at step (a) comprises phage isolates, and more preferably, it comprises a pool of phages isolated in different geographic locations. For instance, virulent lytic phages can be isolated from anywhere bacteria can exist: soil, open water (e.g., oceans, seas, rivers, lakes, etc.), wells, municipal water and waste products (e.g. fecal material, waste water, raw sewage collected in farms, hospitals and/or municipal waste) or from living infected hosts (e.g. body fluids, body cavities, lungs, etc.).

Similarly, it is preferable according to the method of the invention that the pathogenic bacteria contacted at step (a) comprise plurality of different strains of bacteria and or bacteria isolated from a plurality of infected hosts (e.g., humans, animals, birds, fish, plants) living in different geographic locations, and more preferably bacteria originating from a plurality of bacterial clinical isolates. In a preferred embodiment, at step (a) the bacteriophages are contacted simultaneously with a plurality of different strains of pathogenic bacteria. Preferably also, the non-pathogenic bacteria contacted at step (c) comprise plurality of different strains of bacteria and or bacteria isolated from a non-infected host. More preferably, the non-pathogenic bacteria contacted at step (c) comprise bacteria living in symbiosis with a living host (e.g. humans, animals, etc.)

Therefore, according to the method of the present invention, it is possible to select phages based on their ability to efficiently kill defined pathogenic bacteria without affecting related normal bacteria which may be found in the normal flora of a host (mouth, intestine, body cavities, skin, etc). More preferably, this is achieved by checking cross-reaction of isolated phages against 1) a large panel of unique pathogenic bacteria isolated locally from infected hosts and 2) against non-pathogenic bacteria isolated from non-infected hosts. Therefore, only those phages reacting strongly with the pathogenic bacteria and having no effect on the non-pathogenic panel of bacteria are selected. Example 2 hereinafter demonstrates the efficiency of this concept.

D) Selection of Non-Toxic Phages

According to a third major aspect the invention, it is provided methods for evaluating bacteriophage susceptibility to external genetic modifications. The essence of the methods of the invention consists in comparing a restriction digestion pattern of genetic material of phages contacted with a bacteria with a corresponding restriction digest ion pattern from phages not contacted with the same bacteria (i.e. a “control” restriction digestion pattern). A difference between the restriction digestion patterns is thus indicative that the bacteriophages are susceptible to genetic modifications.

According to an embodiment of the method, the user of the method is already in possession of a control restriction digestion pattern. Indeed, it is conceivable that restriction digestion patterns for phages be obtained commercially in the future. In such case the method comprises the steps of:

a) providing bacteriophages for which susceptibility to genetic modifications is to be evaluated;

b) contacting the bacteriophages with bacteria under suitable conditions and for a sufficient period of time to allow the bacteria to cause genetic modifications to the bacteriophages if this type of bacteriophages have such a susceptibility;

c) isolating bacteriophages from the bacteriophages contacted at step b);

d) digesting with a plurality of restriction enzymes, genetic material from the bacteriophages isolated at step c), thereby obtaining a restriction digestion pattern for the isolated bacteriophages; and

e) comparing the restriction digestion pattern of step d) with a control restriction digestion pattern obtained from phages not contacted with the bacteria, wherein a difference between these restriction digestion patterns is indicative that said type of bacteriophage is susceptible to genetic modifications.

According to another embodiment of the method, the user of the method is not in possession of a control digestion pattern. In such case the method comprises the steps of:

a) providing a first and a second pool of bacteriophages, the first and second pools both comprising a plurality of an identical type of bacteriophages for which susceptibility to genetic modifications is to be evaluated;

b) contacting bacteriophages from the first pool with bacteria under suitable conditions and for a sufficient period of time to allow said bacteria to cause genetic modifications to the bacteriophages if this type of bacteriophages have such a susceptibility;

c) isolating bacteriophages from the samples contacted at step b) and c);

d) digesting separately, with a plurality of restriction enzymes, genetic material from the bacteriophages from the second pool and genetic material of bacteriophages isolated at step c), thereby obtaining a restriction digestion pattern for bacteriophages of each one of the first and second pools; and

e) comparing the restriction digestion patterns of step d), wherein a difference between said restriction digestion patterns is indicative that said type of bacteriophages is susceptible to genetic modifications.

Preferably, the method further comprises the step of incubating the second pool of bacteriophages in the absence of bacteria under the conditions defined described in b), prior digesting this second pool of bacteriophages. This will permit to detect and analyze spontaneous modifications of the phage being evaluated.

Genetic modifications that may be detected with the methods of the invention, include but is not limited to deletions, additions, mutations, or recombination in the genetic material of the bacteriophages.

The methods of the invention may also be used detecting induction of a bacteriophage present in a bacteria. Such method comprises the steps of carrying out steps (a) to (e) of a method for evaluating bacterial susceptibility to external genetic modifications as defined previously. However, obtaining a supplementary restriction digestion pattern (i.e. doubled, tripled and more) for the bacteriophages isolated at step c) and digested at step (d), would be indicative that an induction of bacteriophage(s) in the bacteria contacted at step (a) occurred.

Of course, steps (a) to (e) of the methods may be repeated with a different strain bacteria or with a pool of bacteria from a plurality of different strains. Preferably, at step (b), the bacteriophages are contacted with bacteria capable of being infected by the bacteriophages. More preferably, the bacteriophages are contacted with bacteria capable of being lysed by the bacteriophages. Even more preferably, the bacteriophages are contacted with a plurality of different strains of pathogenic bacteria.

In another preferred embodiment, the methods of the invention are carried out to select therapeutic phages selected for their inability to incorporate bacterial genetic sequence coding for toxins. To do so, at step (b) the bacteriophages are contacted with bacteria comprising a genome with a gene coding for a toxin and/or for a virulence factor.

In another preferred embodiment, the methods further comprise the step of selecting for a therapeutic application bacteriophages for which susceptibility to genetic modifications has proven to be substantially nonexistent negative or totally negative.

### EXAMPLES

The following examples are illustrative of the wide range of applicability of the present invention and are not intended to limit its scope. Modifications and variations can be made therein without departing from the spirit and scope of the invention. Although any method and material similar or equivalent to those described herein can be used in the practice for testing of the present invention, the preferred methods and materials are described.

Problematic

Phage therapy is a very interesting approach for reducing the use of antibiotics in humans and livestock. Overuse of antibiotics in these two sectors is one of the key elements for the development of antibiotic resistance.

Use of bacteriophages for treating bacterial infections in livestock will help preserve the use of antibiotics for human use. Phages can also be used for the treatment of bacterial infections in humans, which are caused by antibiotic resistant bacteria. Such infections are on the rise, with very few new antibiotics in the pipeline of the pharmaceutical companies. An important property of bacteriophages that make them ideal candidates for phage therapy applications is their specificity to a targeted bacteria and their inability to infect unrelated bacteria or mammalian cells.

An important advantage of phages is their self-reproduction. Phages self-reproduce as long as corresponding host bacteria are present, so the need to repeatedly administer the phage is greatly reduced.

### Example 1

### Incorporation of a Non-Functional Signature Label in the Genome of a Phase for Monitoring

Objective:

This experiment is carried out to confirm that, phages modified to carry an exogenous nucleotide “signature” or “label” sequence having from 5 to 30 nucleotides, will be useful for the identification, the monitoring and also quantification purposes.

Strategy:

As an example of this modification process, addition of a genetic label in the non-coding region of an E. coli phage, phi-X 174 is presented. The genome of this phage is 5386 bases and made up of circular single stranded DNA. The complete genome of this phage has been sequenced and it codes for 11 proteins (Genbank™ database, Accession # J02482). In this example, the label is introduced in the non-coding region between the putative minor spike protein (2931-3917) and the site for RF replication (3962). Two enzymes Mfel (3939) and ApaLl (4779) are chosen for restriction digestion in this region. The most important characteristic for the choice of the restriction enzymes was their ability to cut the phage genome at a single site. Restriction digestion with these two enzymes produces an 840 bp and a 4.5 Kb fragment. The 840 bp fragment is removed and replaced by a PCR generated fragment containing the tag.

In this example, the label is introduced around the Mfel site. Accordingly, two different oligos are designed:

1) a 3′ oligo (phiXR) which is complimentary to the phi-X 174 phage sequence around the ApaLl site (5′-CATAAAGTGCACCGCATGG-3′; SEQ ID NO: 1); and

2) a 5′ “label” oligo (5′-GAGTAGCMTTGATTCACGCTGTATGTTTT CATGCCTCC-3′; SEQ ID NO: 2; phiXF) consisting of:

i) a short sequence of phiX-174 5′ to the Mfel site and including the restriction site (5′-GAGTAGCAATTG-3′);

ii) a 10 bp “signature sequence” (5′-ATTCACGCTG-3′); and

iii) a sequence from the phiX174 phage 3′ to the Mfel site 5′-TATGTTTTCATGCCTCC-3′).

All DNA manipulations are done using the double stranded RF form of the phage DNA prepared using standard protocols as outlined in Current protocols (Current protocols 1990, chapter 1.15.3). A PCR reaction is performed with the 5′ and 3′ oligos designed earlier, using phage DNA as template and a proof-reading DNA polymerase enzyme. Under these conditions, a 840 bp PCR product is generated. The PCR product is digested with Mfel and ApaLl and the fragment purified from the gel using standard protocols (Gibco, Concert rapid gel extraction system). Phage DNA is also digested with the Mfel and ApaLl enzymes, the 840 bp fragment removed and the 4.5 Kb band purified. The PCR generated and digested fragment is ligated to the 4.5 Kb phage DNA fragment using T4 DNA ligase. The ligation mix is transferred to the host bacteria by electroporation using standard protocols (BiORad Micro Pulser #1652100, program Ec2). The presence of lysis in the sample indicates the presence of infective phages demonstrating that the introduction of the label at this position does not interfere with the normal functions of the phage.

Once the label is introduced, the presence of the labeled phage can be easily monitored by PCR using specific oligos phiXF2—5′-GACCAGGTATATGCAC-3′ (SEQ ID NO: 3) (starting at 3498); and phiXR2—5′-TGAAAACATACAGCGTG-3′ (SEQ ID NO: 4) (finishing at base 3954 of the wild type phage). The generation of a 466 bp band indicates the presence of the label. No band should be observed in the absence of the label.

Phages carrying the label may then be characterized both at the biological and molecular level. These studies may include:

Effect of introduction of the label on the infectivity of the phage particle;

Effect of the label on the stability of the phage;

Host range of the labeled-phage;

Inability to cross react with non-pathogenic bacteria; and

Safety of the phage by performing transduction studies.

### Example 2

### Development of Bacteriophage Libraries for Phase Therapy Applications—Selection of Bacteriophages using a Unique Panel of Bacteria

Objective:

The objective of this experiment was to select and characterize bacteriophages against pathogenic bacteria causing infections in livestock. Phages were isolated from sewage obtained from several swine farms in different geographic locations and selected against a unique panel of pathogenic bacteria isolated from infected animals and non-pathogenic bacteria isolated from the gut of normal animals. Phages showing the best cross-reactivity profile with the pathogenic and no cross-reactivity with the non-pathogenic bacteria were chosen for further development. Phages were further classified based on their unique cross-reactivity, restriction digest and electronic microscopy profile.

Materials and Methods:

Sewage samples were collected from several large swine farms across the provinces of Quebec and Ontario (Canada), and stored at 4° C. until used. The sewage was allowed to react with the pathogenic bacteria under study and incubated overnight. Phages present in the preparation amplified because the host bacteria was present in the media. The amplified bacteriophages were then allowed to interact with fresh host bacteria and the ability of these phages to produce lysis plaques was tested. Only phages that were able to lyse the bacteria were extracted from the plaque plug and were used for further purification. The phages were subjected to 3-4 more rounds of selection as outlined above and the titer of the preparation determined.

The cross-reactivity of the most virulent phages was then extensively tested against a unique panel of pathogenic bacteria isolated from infected animals, as well as a panel of non-pathogenic bacteria isolated from the gut of non-infected animals. This was done by adding a drop of the phage preparation on the desired bacterial lawn. The presence of lysis plaques indicates a cross-reaction. Phages showing no cross-reactivity with the non-pathogenic bacteria but reacting very strongly with some bacteria in the pathogenic panel were developed further. These phage preparations were amplified in liquid culture. The lysate was heated at 58° C. for 30 min and passed through a 0.22 μm filter to remove any bacteria and large bacterial debris. Phages were then concentrated by differential centrifugation. The purified phage preparations were then subjected to Electron Microscopy (EM) to determine their purity and morphological characteristics.

Results:

Phages against several Salmonella and enterotoxigenic E. coli serotypes pathogenic to swine were isolated, purified, selected and analyzed by EM. A representative cross-reactivity data for O64 phages is presented in Table 1. In the case of E. coli, a unique panel consisting of 37 different strains of pathogenic bacteria and 21 different strains of non-pathogenic E. coli was used. All the phages against O64 that were isolated did not show any cross-reactivity with non-pathogenic strains of E. coli. These phages also showed varied cross-reactivity with the pathogenic strains. Data presented in Table 1 were combined with the restriction digest pattern and the EM data for further classification into groups in the phage libraries.

Among the phages that were isolated, two were particularly interesting: bacteriophage LT2 431 against Salmonella typhimurium and phage BTP 1.1 against E. coli serotype O64. Both of these phages showed no cross-reactivity against a panel of non-pathogenic bacteria and showed good virulence against their respective host strains. Electron micrograph studies revealed that, in both cases, the phage particles have a rigid tail with an isometric head of about 60 nm, phage BTP 1.1 having a slightly bigger head, measuring 75-80 nm in diameter and a rigid tail with a sheath (not shown).

TABLE 1
Cross-reactivity of isolated bacteriophages with pathogenic and non-pathogenic strains of E. coli

| | | | | E10- | E13- | E16- | E19- | E22- | E25- | E28- | E31- | E34- | | |
|---|---|---|---|---|---|---|---|---|---|---|---|---|---|---|
| | E1-E3 | E4-E6 | E7-E9 | E12 | E15 | E18 | E21 | E24 | E27 | E30 | E33 | E36 | E37 | N1-N20 |
| BTP-1.1 | | | | +++ | +++ | +++ | ++ | | | | | | | 0 |
| BTP-1.2 | | | + | ++ | ++ | ++ | ++ | + | | + | | | | 0 |
| BTP-1.3 | | | | ++ | ++ | + | ++ | | + | | | | | 0 |
| BTP-1.4 | | | | ++ | ++ | ++ | + | | | + | | | | 0 |
| BTP-1.5 | | | | ++ | ++ | ++ | ++ | | | + | | | | 0 |
| BTP-1.6 | | + | + | + | + | + | ++ | + | + | + | | | | 0 |
| BTP-1.7 | | | | ++ | ++ | + | + | | | | | | | 0 |
| BTP-1.8 | | | | ++ | ++ | ++ | + | | | | | | | 0 |
| BTP-1.9 | | + | + | +++ | ++ | + | + | | + | +++ | + | ++ | + | 0 |
| BTP-1.10 | | | | ++ | ++ | ++ | ++ | | | | | | | 0 |
| BTP-1.11 | | | | ++ | ++ | ++ | ++ | + | | + | | | | 0 |
| BTP-1.12 | | | | ++ | ++ | ++ | ++ | | | + | | | | 0 |
| BTP-1.13 | | + | | ++ | ++ | + | + | | | | | | | 0 |
| BTP-1.14 | | | | ++ | ++ | + | ++ | | | | | | | 0 |
| BTP-1.15 | | | + | ++ | + | + | ++ | | + | | | | | 0 |
| BTP-1.16 | | | | ++ | ++ | + | ++ | | | + | | | | 0 |
| BTP-1.17 | | | | ++ | ++ | ++ | ++ | + | | + | | | | 0 |
| BTP-1.18 | | | | + | | | ++ | | | | | | | 0 |

E1-E37: Pathogenic E. coli strains isolated from infected animals
N1-N21: Non-Pathogenic E. coli strains isolated from the gut of non-infected animals

The efficacy of one of these two phages (phage BTP 1.1), in the treatment of bacterial enteritis in newborn pig, was tested. Newborn piglets were infected with an enterotoxigenic strain of E. coli O64 which causes severe diarrhea. Treatment of the infected animals with phage BTP 1.1 cured the animals of their symptoms within 48 hrs (not shown). When the phages were used as a prophylactic (given 3 hrs before bacterial challenge), the diarrhea produced was not as severe as that in the untreated group. These animals are also cured of the diarrhea in 48 hrs after challenge with the pathogen. In the untreated group, the animals continued to have diarrhea even after 4 days.

### Example 3

### Selection and Characterization of Therapeutic Non Toxic Phases

Objective:

This experiment was carried out for confirming the absence of genetic sequence coding for toxins in the phages isolated and selected in Example 2. This experiment comprises novel and unique transduction studies that are performed in order to select phages that are not susceptible to incorporate genetic sequences transferred from other related pathogenic organisms, such as toxic bacteria.

Material and Methods:

i) Restriction Digests:

The genome of most, if not all, the phages isolated in the laboratories of the Applicant is made of double-stranded DNA. The restriction digestion pattern of a Salmonella phage isolated in-house is presented as an example. Phage DNA was prepared by standard PEG precipitation protocols (Current protocols in molecular biology, 1995, chapter 1.13). DNA (0.5 μg) was digested with the following restriction endonucleases using manufacturer recommended protocols: EcoRI, EcoRV, HindIII, PstI and SalI (New England Biolabs enzyme data sheet).

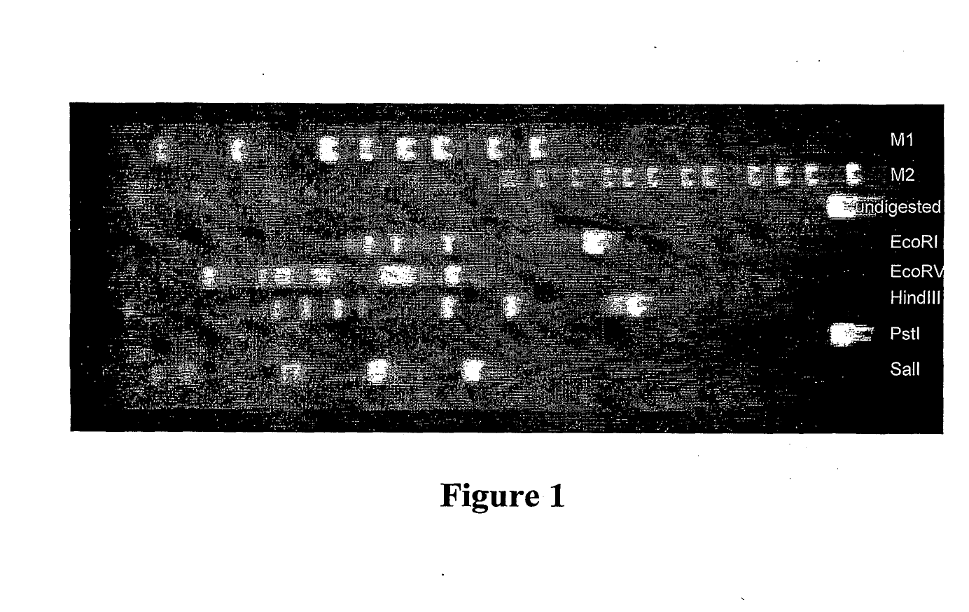

The restriction digest pattern obtained is presented in FIG. 1. Briefly, phage DNA (0.5 μg) was digested with EcoRI, EcoRV, HindIII, PstI and SalI according to manufacturer recommended protocol. The digests were analyzed on a FIGE™ mapper (BiORad laboratories) using a 1% agarose gel and using program U2™ on the instrument. Undigested phage DNA was also loaded for comparison purposes.

As shown in FIG. 1, distinct patterns were obtained with each of the enzymes used except PstI which did not digest the phage DNA. This distinct pattern can be used for the identification of Salmonella DT108 specific bacteriophage. Consequently, in the course of any particular manipulation, any change to this pattern can be easily identified.

The restriction digestion pattern can also be used to classify the different phages in any therapeutic phage library. The classification can be done using the restriction digestion pattern from more than one enzyme and is a powerful tool to identify similar phages early on, in the purification protocol.

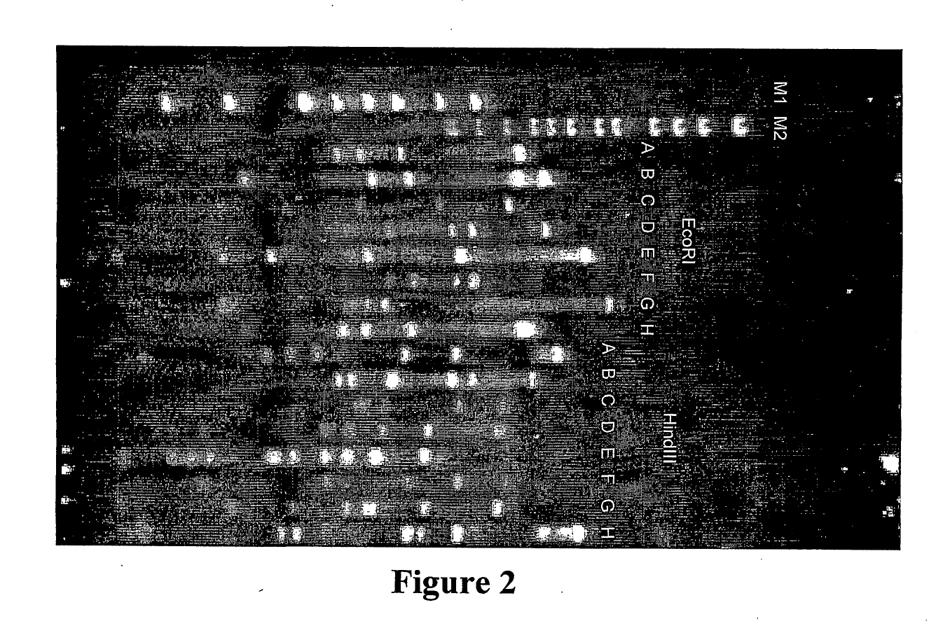

As an example, the restriction digestion pattern of eight salmonella phages with two different restriction enzymes EcoRI and HindIII is presented in FIG. 2. The use of EcoRI and HindIII gave very distinct patterns for these 8 classes of Salmonella phages illustrating that this approach can be the basis of a novel phage classification. The distinct restriction pattern obtained for a particular phage with a set of restriction enzymes can be used in following any changes to the genome in transduction studies.

ii) Sequencing:

Phages, and more particularly those showing a high killing efficacy against a pathogenic bacteria, may be sequenced to confirm the presence or absence of (a) any known toxin genes in their phage genome, (b) regions of homology to toxin genes where these genes can integrate and (c) confirm the absence of elements required for lysogeny.

Typically, the DNA of each of the bacteriophages is digested with a panel of restriction enzymes. Restriction fragments are generated and cloned into cloning vectors according to standard molecular biology protocols (Current protocols in molecular biology, 1995, chapter 3). The cloned fragments are then sequenced using an automated DNA sequencer (ABI sequencer™ 370-stretch). The generated data are analyzed using a sequence analysis program to determine Open Reading Frames (ORFs) (Vector NTI™, Informax Inc.). The translated amino acid information generated is then used to perform homology searches using different search engines available in the public domain, to determine its potential function (eg: http://searchlauncher.bcm.tmc.edu/;http://pubmed).

iii) Transduction Studies:

In order to prove the inability of the isolated bacteriophages to integrate toxin genes, the bacteriophage under study were co-cultured with a related bacteria known to carry these toxin genes. Indeed, the Applicant submit that verification of the ability of a phage to either acquire new DNA or lose portions of genomic DNA when incubated with other related and unrelated bacteria, is a very important aspect to be considered for determining the suitability of a phage for therapeutic uses. The approach presented here is quick and can be easily implemented with any given bacteria or bacterial pool.

As an example, a Salmonella DT108 specific bacteriophage was co-cultured with a pool of E. coli commonly infecting swine (O149, O139), E. coli O157:H7 and 2 serovars of Salmonella typhimurium. After an overnight incubation, the bacteria were removed and the phages amplified using the host DT108 bacteria. The newly amplified phages were used to infect a fresh bacterial pool as outlined above. This process was repeated for seven consecutive days. At the end of the experimental period, phages were isolated, and the phage DNA was purified according to standard protocols. The DNA was digested with an endonuclease, HaelI, to verify if the phage genome had either gained or lost any genomic DNA during the incubation process. As a control, Salmonella DT108 specific bacteriophage was incubated under the same conditions but in the absence of the bacterial pool.

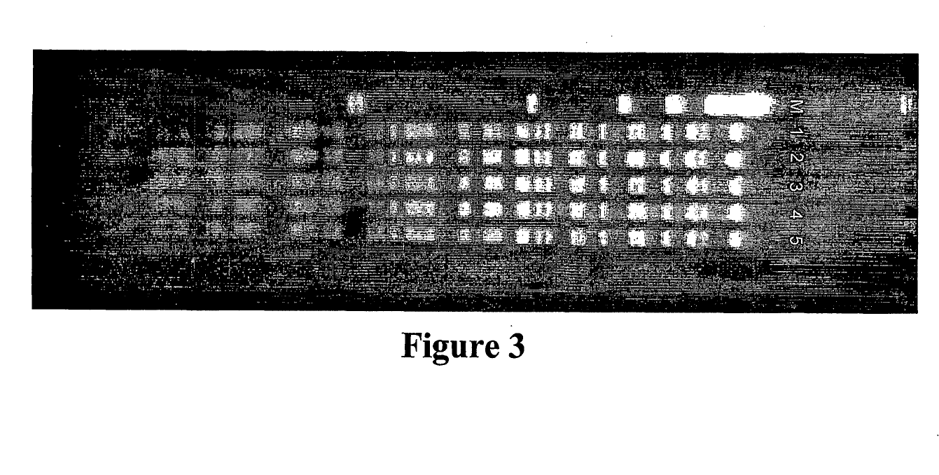

The restriction digestion pattern of both phages obtained is shown in FIG. 3. DNA samples obtained on days 5 and 7 of the transduction study were analyzed and compared with the control pattern of Salmonella DT108 specific bacteriophage. The patterns obtained with the samples from the transduction study were identical to those observed with the control phage incubated under the same conditions. These data demonstrate that the phage genome has neither lost nor gained any DNA during the process of incubation with the pool of pathogenic bacteria.

While several embodiments of the invention have been described, it will be understood that the present invention is capable of further modifications, and the present patent application is intended to cover any variations, uses, or adaptations of the invention, following in general the principles of the invention and including such departures from the present disclosure as to come within knowledge or customary practice in the art to which the invention pertains and as may be applied to the essential features hereinbefore set forth and falling within the scope of the invention or the limits of the appended claims.

1. A genetically labeled bacteriophage, the bacteriophage having a genetically modified genome comprising an exogenous nucleic acid label sequence, said label sequence consisting of an assemblage of nucleotides forming a detectable signature sequence having a non-functional coding function.

2. The bacteriophage of claim 1, wherein said exogenous nucleic acid label sequence is inserted in a non-coding region of the genome of the bacteriophage.

3. The bacteriophage of claim 1 or 2, wherein said exogenous nucleic acid label sequence comprises from about 5 to about 500 nucleotides.

4. The bacteriophage of claim 3, wherein said exogenous nucleic acid label sequence comprises from about 10 to about 50 nucleotides.

5. The bacteriophage of claim 4, wherein said exogenous nucleic acid label sequence comprises from about 10 to about 25 nucleotides.

6. The bacteriophage of claim 1, wherein said exogenous nucleic acid label consist of DNA or RNA.

7. The bacteriophage of claim 1, comprising a plurality of exogenous nucleic acid label sequences.

8. The bacteriophage of claim 1, wherein said bacteriophage consist of a bacteriophage capable of infecting a bacterial organism selected from the group consisting of: Actinobaciltii, Actinomyces, Aeromonas, Archaebacteria, Agrobacteria, Aromabacter, Bacilli, Bacteriodes, Bifidobacteria, Bordetella, Borrelii, Brucella, Burkholderia, Calymmatobacteria, Campylobacter, Capnocytophaga, Citrobacter, Chlamydia, Clostridium, Coccus, Coprococci, Corynebacterium, Cyanobacter, Enterobacter, Enterococci, Eubacteria, Escherichia, Francisella, Helicobacter, Hemophilii, Hidradenitis suppurativa, Lactobacilli, Lawsonia, Legionella, Leptospirex, Listeria, Klebsiella, Mycobacterium, Mobiluncus, Neisserii, Nomerans, Pasteurella, Prevotella, Pneumococci, Propionibacteria, Proteus, Pseudomonas, Pyrococci, Salmonella, Selemonas Serratia, Shigella, Streptococci, Staphylococci, Streoticiccys, Succinimonas, Treponema, Veillonella, Vibrio, Wolinella, Xanthomonas, and Yersinia.

9. A method for producing a genetically labeled bacteriophage as defined in claim 1, comprising the steps of:
providing a biologically active bacteriophage with a genome;
providing an exogenous detectable nucleic acid sequence to be used as a label; and
inserting said exogenous detectable nucleic acid sequence into a suitable region of the genome of the bacteriophage so that said insertion does not substantially negatively affect said bacteriophage biological activity.

10. A method for producing a genetically labeled bacteriophage which can be distinguished from non-labeled bacteriophages, comprising the steps of:
providing a biologically active bacteriophage with a genome;
providing an exogenous detectable nucleic acid sequence to be used as a label, said label sequence consisting of an assemblage of nucleotides forming a detectable signature sequence having a non-functional coding function; and
inserting said exogenous detectable nucleic acid sequence into a suitable region of the genome of the bacteriophage so that said insertion does not substantially negatively affect said bacteriophage biological activity;
whereby detection of said exogenous detectable nucleic acid sequence allows to distinguish genetically labeled bacteriophages from non-labeled bacteriophages.

11. The method of claim 10, wherein said exogenous detectable nucleic acid sequence is inserted in a non-coding region of the bacteriophage genome.

12. The method of claim 10 or 11, said exogenous detectable nucleic acid sequence comprises from about 5 to about 500 nucleotides.

13. The method of claim 12, wherein said exogenous detectable nucleic acid sequence comprises from about 10 to about 50 nucleotides.

14. The method of claim 13 wherein said exogenous detectable nucleic acid sequence comprises from about 10 to about 25 nucleotides.

15. The bacteriophage of claim 10, wherein said exogenous nucleic acid label consist of DNA or RNA.

16. The method of claim 10, wherein a plurality of exogenous detectable nucleic acid sequences are inserted into the genome of the bacteriophage.

17. The method of claim 10, wherein said exogenous detectable nucleic acid sequence is detectable using detection means selected from the group consisting of PCR assays, sequencing, hybridization, high-throughput simultaneous test arrays, gel-shift assays, dyes and labels.

18. A bacteriophage which has been produced according to the method of claim 9.

19. A method of phage therapy, wherein labeled bacteriophages as defined in claim 1 are administered to a host.

20. A method for determining or monitoring the presence or absence of bacteriophages in a sample, comprising the step of detecting the presence in said sample of labeled bacteriophages as defined in claim 1.

21. A method for the control of a bacterial infection or contamination in a host, comprising the step of administering to said host a plurality of biologically active and genetically modified bacteriophages, each of said bacteriophages having a genome genetically modified to comprise an exogenous nucleic acid label sequence consisting of an assemblage of nucleotides forming a detectable signature sequence having a non-functional function.

22. The method of claims 21, further comprising the step of monitoring the presence of said bacteriophages in said host.

23. The method of claim 22, wherein said monitoring step consists of detecting said exogenous nucleic acid label sequence.

24. The method of claim 23, wherein said exogenous detectable nucleic acid sequence is detectable using detection means selected from the group consisting of PCR assays, sequencing, hybridization, high-throughput simultaneous test arrays, gel-shift assays, dyes and labels.

25. The method of any one of claims 21 to 24, wherein said host is selected from the group consisting of mammals, fish, sea food, birds, insects, invertebrates, reptiles, algae, and plants.

26. The method of claim 25, wherein said mammal is selected from humans, livestock, domestic animals, and wild animals.

27. The method of any one of claims 21 to 24, wherein said host consist of a slaughtered animal which has been raised for human consumption purposes, and wherein said monitoring step is performed on sample taken from the carcass of said slaughtered animal.

28. The method of claim 27, wherein said biologically active and genetically modified bacteriophages are administered to the animal about 20 days to about 6 hours before slaughtering.

29. The method of claim 27, wherein said sample is a sample taken from the group consisting of body fluids, solid organs, muscles, body cavities and skin samples.

30. A method for confirming the absence of bacteriophages in an animal carcass derived from a living animal subjected to a treatment of phage therapy with labeled bacteriophages as defined in claim 1, the method comprising the steps of:
obtaining a sample from said carcass; and
assaying said sample for detecting the presence or absence of said labeled bacteriophages;
whereby, absence of detection for said bacteriophages is indicative that said carcass is free from said bacteriophages.

31. The method of claim 30, wherein the detection consists of detecting the exogenous nucleic acid label sequence of the labeled bacteriophages.

32. The method of claim 30 or 31, wherein said sample is a sample taken from the group consisting of body fluids, solid organs, muscles body cavities and skin.

33. The method of claim 30, wherein said animal is selected from the group consisting of pigs, cattle, chicken, turkey, rabbits, horses, and wild animals.

34. A method for selecting bacteriophages capable of lysing a selected strain of pathogenic bacteria without lysing bacteria from a selected non-pathogenic strain, the method comprising the steps of:
a) contacting under conditions suitable for lysis: i) bactedophages capable of lysing a selected pathogenic strain of bacteria, and ii) bacteria from said selected pathogenic strain;
b) isolating, from the bacteriophages of step a), bacteriophages capable of lysing at least some of said pathogenic bacteria;
c) contacting, under conditions suitable for lysis, bacteriophages isolated at step b) with a selected strain of non-pathogenic bacteria for which lysis is undesirable; and
d) selecting, from the bacteriophages of step c), bacteriophages which do not lyse more than 5% of the non-pathogenic bacteria they have been contacted with.

35. The method of claim 34, wherein said pool of bacteriophages comprises bacteriophages isolated from the group consisting of living infected hosts, soil, water and waste.

36. The method of claim 34 or 35, wherein the pathogenic bacteria contacted at step a) comprise bacteria isolated from a plurality of infected hosts living in different geographic locations.

37. The method of claim 34, wherein the pathogenic bacteria contacted at step a) comprise bacteria from a plurality of bacterial clinical isolates.

38. The method of claim 34, wherein the non-pathogenic bacteria contacted at step c) comprise bacteria isolated from non-infected hosts from different geographic locations.

39. The method of claim 34, wherein the non-pathogenic bacteria contacted at step c) comprise bacteria living in symbiosis with a living host.

40. The method of claim 34, wherein at step a) the bacteriophages are contacted simultaneously with a plurality of different strains of pathogenic bacteria.

41. The method of claim 34, wherein at step c) the bacteriophages are contacted simultaneously with a plurality of different strains of non-pathogenic bacteria.

42. A method for evaluating bacteriophage susceptibility to external genetic modifications, the method comprising the steps of:
a) providing a first and a second pool of bacteriophages, said first and second pools both comprising a plurality of an identical type of bacteriophages for which susceptibility to genetic modifications is to be evaluated;
b) contacting bacteriophages from the first pool with bacteria under suitable conditions and for a sufficient period of time to allow said bacteria to cause genetic modifications to the bacteriophages if said type of bacteriophages have such a susceptibility;
c) isolating bacteriophages from the samples contacted at steps b) and c);
d) digesting separately genetic material from the bacteriophages from the second pool and genetic material of bacteriophages isolated at step c) with a plurality of restriction enzymes, thereby obtaining a restriction digestion pattern for bacteriophages of each one of the first and second said pools; and
e) comparing the restriction digestion patterns of step d), wherein a difference between said restriction digestion patterns is indicative that said type of bacteriophages is susceptible to genetic modifications.

43. The method of claim 42, wherein at step b), bacteriophages are contacted with bacteria capable of being infected by said bacteriophages.

44. The method of claim 42 or 43, wherein at step b), bacteriophages are contacted with bacteria capable of being lysed by said bacteriophages.

45. The method of claim 42, wherein at step b), bacteriophages are contacted with a plurality of different strains of pathogenic bacteria.

46. The method of claim 42, wherein steps a) to e) are repeated with a different strain bacteria or with a pool of bacteria from a plurality of different strains.

47. The method of claim 42; further comprising the step of incubating the second pool of bacteriophages in the absence of bacteria under the conditions defined described in b), prior digesting said second pool of bacteriophages.

48. The method of claim 42, wherein said genetic modification is selected from the group consisting of deletions, additions, mutations, or recombination in the genetic material of the bacteriophages.

49. The method of claim 42, wherein said bacteria comprises a genome with a gene coding for a toxin and/or for a virulence factor.

50. The method of claim 42, further comprising the step of selecting for a therapeutic application bacteriophages for which susceptibility has proven to be negative.

51. A method for evaluating bacteriophage susceptibility to external genetic modifications, the method comprising the steps of:
a) providing bacteriophages for which susceptibility to genetic modifications is to be evaluated;
b) contacting said bacteriophages with bacteria under suitable conditions and for a sufficient period of time to allow said bacteria to cause genetic modifications to the bacteriophages if said type of bacteriophages have such a susceptibility;
c) isolating bacteriophages from the bacteriophages contacted at step b);
d) digesting with a plurality of restriction enzymes, genetic material from the bacteriophages isolated at step c), thereby obtaining a restriction digestion pattern for said isolated bacteriophages; and
e) comparing the restriction digestion pattern of step d) with a control restriction digestion pattern obtained from phages not contacted with said bacteria, wherein a difference between said restriction digestion patterns is indicative that said type of bacteriophage is susceptible to genetic modifications.

52. A method for detecting induction of a bacteriophage present in a bacteria, comprising the steps of carrying out steps (a) to (e) of a method as defined in claim 42, with the proviso that obtaining a supplementary restriction digestion pattern for the bacteriophages isolated at step c) and digested at step (d) is indicative of a bacteriophage induction in the bacteria contacted at step (a).


SEQUENCE LISTING (4 sequences)

SEQ ID NO: 1 - DNA, 19 bp, Artificial Sequence
  oligonucleotide
cataaagtgcaccgcatgg

SEQ ID NO: 2 - DNA, 39 bp, Artificial Sequence
  oligonucleotide
gagtagcaattgattcacgctgtatgttttcatgcctcc

SEQ ID NO: 3 - DNA, 16 bp, Artificial Sequence
  oligonucleotide
gaccaggtatatgcac

SEQ ID NO: 4 - DNA, 17 bp, Artificial Sequence
  oligonucleotide
tgaaaacatacagcgtg